(19) United States
(12) Patent Application Publication
(10) Pub. No.: US 2007/0038174 A1
(43) Pub. Date: Feb. 15, 2007

(54) Ophthalmic injector system

(76) Inventor: Mark A. Hopkins, Mission Viejo, CA (US)

Correspondence Address: ALCON, IP LEGAL, TB4-8, 6201 SOUTH FREEWAY, FORT WORTH, TX, 76134, US

(21) Appl. No.: 11/200,452

(22) Filed: Aug. 9, 2005

(51) Int. Cl. A61M 37/00

(52) U.S. Cl. 604/23

(57) ABSTRACT

An ophthalmic injector system having an injection chamber, a dispensing lumen, an actuation chamber, a fluid reservoir, a source of repeating pulses of pressurized gas, and a computer.

### FIELD OF THE INVENTION

The present invention generally pertains to fluid delivery and more particularly to fluid delivery associated with ophthalmic surgery and ophthalmic drug delivery.

### DESCRIPTION OF THE RELATED ART

During ophthalmic surgery, a need exists to inject fluids into the eye at very precise volumes and flow rates. Such injections are typically manually made using a conventional syringe and needle. The surgeon is required to puncture the eye tissue with the needle, hold the syringe steady, and actuate the syringe plunger (with or without the help of a nurse) to inject the fluid into the eye. The volume injected (e.g. about 0.1 cc for sub-retinal fluid injection) is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. Examples of fluids that may need to be injected into the eye during ophthalmic surgery include short-term retinal tamponades (e.g. perflourocarbon liquid) and long-term retinal tamponades (e.g. silicone oil, air/perflourocarbon gas mixture) that are used in the repair of retinal detachments or tears. In addition, a variety of drugs may need to be applied topically to or injected into the eye before, during, or after ophthalmic surgery (e.g. anti-infectives, anti-inflammatories, anti-infective/anti-inflammatories).

Several diseases and conditions of the posterior segment of the eye continue to threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples. Manual injection via a conventional syringe, plunger, and needle is often used to deliver drugs to the vitreous through the pars plana region of the eye to treat some of these conditions.

One commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses a surgical system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

Despite the above-referenced solutions, a need continues to exist for improved ophthalmic fluid delivery.

### SUMMARY OF THE INVENTION

In one aspect, the present invention is an ophthalmic injector system including an injection chamber, a dispensing lumen, an actuation chamber, a fluid reservoir, a source of repeating pulses of pressurized gas, and a computer. The injection chamber is for receiving a first volume of fluid. The dispensing is lumen fluidly coupled to the injection chamber. The actuation chamber contains a separating member. The separating member has a first end fluidly sealed to the actuation chamber and a second end fluidly sealed to the injection chamber. The fluid reservoir is fluidly coupled to the injection chamber and contains the fluid to be injected. The source of repeating pulses of pressurized gas is fluidly coupled to the first end of the separating member. The computer is for controlling the pulse rate of the repeating pulses. During operation of the injector, the computer uses the repeating pulses to repeatedly actuate the separating member to repeatedly displace the first volume of the fluid from the injection chamber and through the dispensing lumen until a desired volume of the fluid has been displaced from the dispensing lumen into an eye.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a schematic illustration of an ophthalmic injector system according to a preferred embodiment of the present invention; and

FIG. 2 is a schematic illustration of an ophthalmic injector system according to a second preferred embodiment of the present invention.

### DETAILED DESCRIPTION OF THE PREFERRED EMBODIMENTS

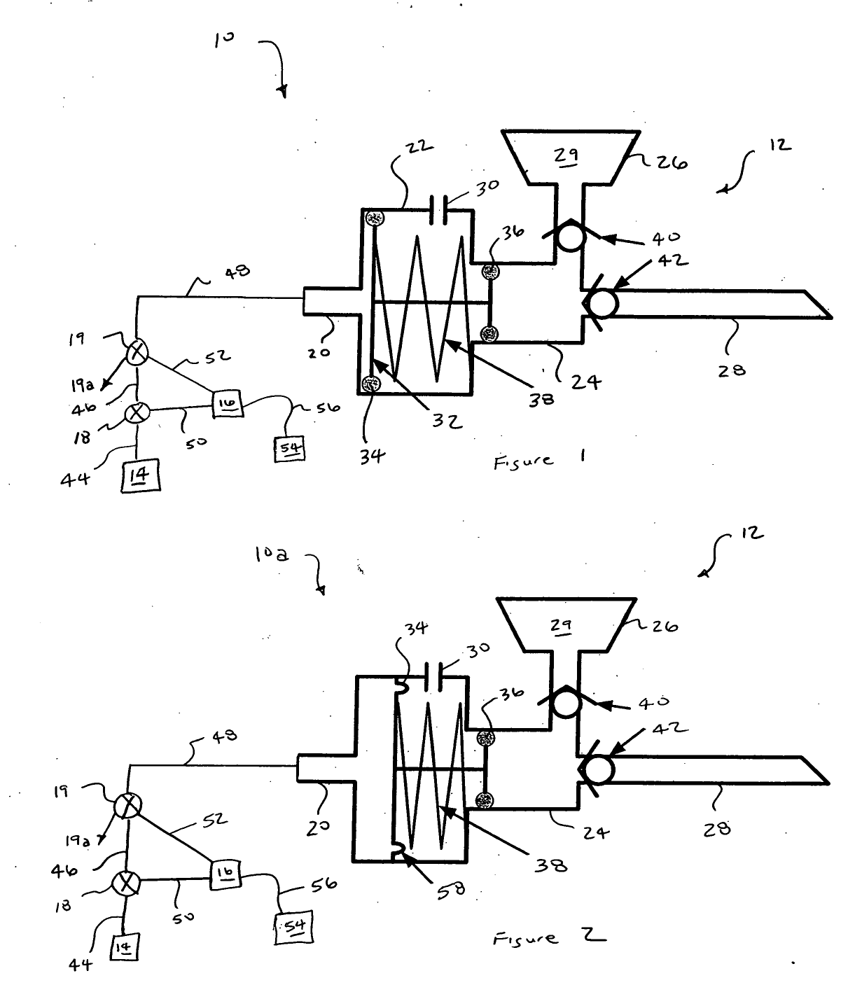

The preferred embodiments of the present invention and their advantages are best understood by referring to FIGS. 1-2 of the drawings, like numerals being used for like and corresponding parts of the various drawings.

Ophthalmic injector system 10 generally includes an injector 12, a pressurized gas source 14, a computer or microprocessor 16, a proportional solenoid valve 18, and an isolation (“on/off”) solenoid valve 19. Injector 12 includes a port 20, an actuation chamber 22, an injection chamber 24, a fluid reservoir 26 fluidly coupled to injection chamber 24, and dispensing lumen 28 fluidly coupled to injection chamber 24. Actuation chamber 22 has an atmospheric vent 30. A separating member 32 is slidably disposed in actuation chamber 22 and injection chamber 24. As shown in FIG. 1, separating member 32 is a piston. Piston 32 has a proximal end 34 that is fluidly sealed to actuation chamber 22 and a distal end 36 that is fluidly sealed to injection chamber 24. A return spring 38 biases piston 32 toward port 20. A one-way valve 40 allows fluid flow from fluid reservoir 26 into injection chamber 24 but not the opposite fluid flow. A one-way valve 42 allows fluid flow from injection chamber 24 into needle 28 but not the opposite fluid flow. Pressurized gas source 14 preferably provides pressurized air. Tubing or manifold 44 fluidly couples pressurized gas source 14 and proportional valve 18, tubing or manifold 46 fluidly couples proportional valve 18 and isolation valve 19, and tubing or manifold 48 fluidly couples isolation valve 19 and port 20. An interface 50 electrically couples microprocessor 16 and proportional valve 18, and an interface 52 electrically couples microprocessor 16 and isolation valve 19. Isolation valve 19 preferably is a three-way valve having an atmospheric vent 19a.

Fluid reservoir 26 may be integral to injector 12, or fluid reservoir 26 may be a cartridge or container that is removably coupled to injector 10. Fluid reservoir holds a fluid 29. Fluid 29 may be any ophthalmically acceptable fluid. For example, fluid 29 may be an intraocular irrigating solution, such as BSS PLUS® intraocular irrigating solution available from Alcon Laboratories, Inc. As another example, fluid 29 may be a short-term or long-term retinal tamponade. As a further example, fluid 29 may include any ophthalmically acceptable drug. Preferred drugs are ophthalmically acceptable drugs for the treatment or prevention of a disease or condition of the posterior segment of the eye, including age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies. Fluid 29 may also include ophthalmically acceptable excipients. Dispensing lumen 28 is preferably a standard, luer-connected, stainless steel needle or cannula. Alternatively, dispensing lumen 28 may be integrated into injector 12.

In operation, a nurse fluidly couples ophthalmic injector 12 to tubing 48 via port 20. Injection chamber 24 and needle 28 are primed with fluid 29. A surgeon or nurse inputs the desired volume and flow rate of fluid 29 to be injected into the eye into microprocessor 16 via an input controller 54. An interface 56 electrically couples microprocessor 16 and input controller 54. The surgeon grasps injector 12 and inserts needle 28 into the target tissue in the eye of a properly anesthetized patient. The surgeon initiates delivery of fluid 29 via another input to microprocessor 16 from input controller 54. Input controller 54 may be any conventional control but preferably includes a touch screen, a foot switch, or both a touch screen and a foot switch. Having input controller 54 include a foot switch is preferred, as this allows the surgeon to use both hands to position injector 12 and hold it steady during fluid delivery.

Upon initiation of fluid delivery, microprocessor 16 opens isolation valve 18 using a signal transferred via interface 50. Pressurized gas source 14 provides pressurized gas to isolation valve 19 via manifolds 44 and 46. Microprocessor 16 opens and closes isolation valve 19 using signals transferred via interface 52 to create repeating pulses of pressurized gas at a desired pulse rate. The pulses of pressurized gas are delivered to piston 32 via tubing 48 and port 20.

For each pulse of pressurized gas, piston 12 is actuated toward needle 28, compressing return spring 38, venting pressure within actuation chamber 22 via vent 30, and displacing the fluid 29 in injection chamber 24 through valve 42 and needle 28 into the eye. Valve 40 prevents fluid 29 in injection chamber 24 from flowing into fluid reservoir 26. After fluid 29 is displaced from needle 28, return spring 38 returns piston 32 to the position shown in FIG. 1, pulling fluid 29 from fluid reservoir 26 through valve 40 to refill injection chamber 24, and venting pressurized gas via vent 19a. Valve 42 prevents fluid 29 in needle 28 from flowing back into injection chamber 24. Microprocessor 16 sets the pulse rate of pressurized gas based upon the desired flow rate of fluid 29 from needle 28 into the eye. The desired volume of fluid to be injected into the eye is proportional to the volume of injection chamber 24. Microprocessor 16 continues the repeating pulses of pressurized gas until the desired volume of fluid is injected into the eye. Injection chamber 24 has a volume small enough to meet the minimum resolution required for the specific application of ophthalmic injector system 10. The volume of fluid 29 within needle 28 after each pressure pulse is very small compared to the volume of injection chamber 24 and is retained within needle 28 via the surface tension of fluid 29.

FIG. 2 shows an ophthalmic injector system 10a that is identical to ophthalmic injector system 10, with the exception that separating member 32 is a diaphragm 58 instead of a piston. The operation of injector system 10a to inject a precise volume of fluid 29 into the eye at a desired flow rate is substantially identical to that described above for injector system 10.

From the above, it may be appreciated that the present invention provides improved devices and methods for safe, effective, delivery of fluid to the eye, and particularly to the posterior segment of the eye. The present invention allows a surgeon to inject fluid into the eye at precise volumes and flow rates regardless of the properties of the fluid (e.g. density, viscosity, temperatures). The present invention is illustrated herein by example, and various modifications may be made by a person of ordinary skill in the art. For example, while the present invention is described above in connection with an intraocular injection of fluid, it is equally applicable for topical application of fluid to the eye.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. An ophthalmic injector system, comprising:
an injection chamber for receiving a first volume of fluid;
a dispensing lumen fluidly coupled to said injection chamber;
an actuation chamber containing a separating member, said separating member having a first end fluidly sealed to said actuation chamber and a second end fluidly sealed to said injection chamber;
a fluid reservoir fluidly coupled to said injection chamber and containing said fluid;
a source of repeating pulses of pressurized gas fluidly coupled to said first end of said separating member; and
a computer for controlling the pulse rate of said repeating pulses;
whereby said computer uses said repeating pulses to repeatedly actuate said separating member to repeatedly displace said first volume of said fluid from said injection chamber and through said dispensing lumen until a desired volume of said fluid has been displaced from said dispensing lumen into an eye.

2. The ophthalmic injector system of claim 1 wherein said computer uses said pulse rate to control a flow rate of said fluid displaced from said dispensing lumen into said eye.

3. The ophthalmic injector system of claim 1 wherein said separating member is a piston.

4. The ophthalmic injector system of claim 1 wherein said separating member is a diaphragm.

5. The ophthalmic injector system of claim 1 wherein said dispensing lumen is a needle.

6. The ophthalmic injector system of claim 1 wherein said dispensing lumen is a cannula.

7. The ophthalmic injector system of claim 1 wherein:
said injection chamber, said dispensing lumen, said actuation chamber, and said fluid reservoir are disposed in an injector; and
said source of repeating pulses of pressurized gas and said computer are disposed external to said injector.

8. The ophthalmic injector system of claim 7 wherein said fluid reservoir is integrally formed in said injector.

9. The ophthalmic injector system of claim 7 wherein said fluid reservoir is removably coupled to said injector.

10. The ophthalmic injector system of claim 1 wherein said source of repeating pulses of pressurized gas comprises:
a pressurized gas source;
a proportional valve fluidly coupled to said pressurized gas source and electrically coupled to said computer; and
an isolation valve fluidly coupled to said pressurized gas source and electrically coupled to said computer.

11. The ophthalmic injector system of claim 10 wherein said source of repeating pulses of pressurized gas comprises an input controller.

12. The ophthalmic injector system of claim 11 wherein said input controller comprises a foot switch.

13. The ophthalmic injector system of claim 11 wherein said input controller comprises a touch screen.
